(19) United States
(12) Patent Application Publication
(10) Pub. No.: US 2021/0275622 A1
(43) Pub. Date: Sep. 9, 2021

(54) METHODS OF INCREASING MUSCULAR STRENGTH AND MUSCULAR ENDURANCE WITH ASPARAGUS RACEMOSUS

(71) Applicant: Natreon, Inc., New Brunswick, NJ (US)

(72) Inventor: Sanyasi R. Kalidindi, Monroe, NJ (US)

(73) Assignee: Natreon, Inc., New Brunswick, NJ (US)

(21) Appl. No.: 17/118,956

(22) Filed: Dec. 11, 2020

Related U.S. Application Data
(60) Provisional application No. 62/946,577, filed on Dec. 11, 2019.

(51) Int. Cl. A61K 36/8965; A61P 21/06; A23L 33/105; A23L 33/00

(52) U.S. Cl. CPC A61K 36/8965; A61P 21/06; A23V 2002/00; A23L 33/40; A23L 33/105

(57) ABSTRACT

The present invention is directed to methods of increasing muscular strength and muscular endurance with a combination of the administration of Asparagus racemosus to a subject and resistance training of the subject's body.

### CROSS-REFERENCE TO RELATED APPLICATIONS

This application claims the benefit of U.S. Provisional Application No. 62/946,577, filed Dec. 11, 2019. The provisional application is incorporated by reference in its entirety herein.

### FIELD OF INVENTION

This invention relates to methods of increasing muscular strength and/or muscular endurance with a combination of the administration of Asparagus racemosus to a subject and resistance training of the subject's body.

### BACKGROUND

Asparagus racemosus is a popular plant in the ancient Ayurvedic tradition that is believed to improve vitality, immunity, and vigor [Govindarajan et al., “Antioxidant approach to disease management and the role of ‘Rasayana’ herbs of Ayurveda” J. Ethnopharmacol. 99:165-178 (2005); Rege et al., “Adaptogenic properties of six rasayana herbs used in Ayurvedic medicine” Phytother. Res. 13:275-291 (1999)]. Asparagus racemosus has previously been demonstrated to elicit antitussive, antibacterial, antihepatotoxic, immunomodulatory, and antioxidant effects in both rat and human models [Alok et al., “Plant profile, phytochemistry and pharmacology of Asparagus racemosus (Shatavari): A review” Asian Pac. J. Trop. Dis. 3:242-251 (2013)]. The primary phytochemicals found in Asparagus racemosus include saponins, such as shatavarin VI and shatavarin VII [Singh et al., “Saponins in pulses and their health promoting activities: A review” Food Chem. 233:540-549 (2017)], as well as antioxidants such as asparagamine A, racemosol, and racemofuran [Bopana et al., “Asparagus racemosus—Ethnopharmacological evaluation and conservation needs” J. Ethnopharmacol. 110:1-15 (2007)]. No studies have investigated the ergogenic potential of Asparagus racemosus.

### BRIEF DESCRIPTION OF THE DRAWINGS

FIG. 1 is a copy of an FTIR (Fourier Transform Infrared) chromatogram of a standardized powdered aqueous extract of Asparagus racemosus of this invention.

FIG. 2 shows a timeline for the double-blind, placebo-controlled investigation described in the Example of the present application.

FIG. 3 is a graph showing the rate of change in bench press training loads is significantly greater with Asparagus racemosus administration than placebo.

### SUMMARY OF INVENTION

The present invention is directed to increasing muscular strength and/or muscular endurance through a combination of administration of Asparagus racemosus and performance of resistance training in a subject, preferably a human subject.

In an embodiment, the present invention is directed to a method of increasing muscular strength and/or muscular endurance in a subject comprising the steps of (a) providing a composition comprising an Asparagus racemosus extract; and (b) administering an effective amount of the composition to the subject to deliver the Asparagus racemosus to the subject's bloodstream and bodily tissues; wherein the subject performs an effective amount of resistance training; and wherein the Asparagus racemosus acts in said bloodstream and/or bodily tissues in combination with said resistance training to increase the subject's muscular strength and muscular endurance.

### DETAILED DESCRIPTION

The present methods are directed to combining Asparagus racemosus administration with resistance training to significantly increase muscular strength and/or endurance. The administration of Asparagus racemosus in combination with resistance training significantly increases muscular strength, for instance as shown in the Example below, where bench press one-repetition maximum (1RM) significantly increased with Asparagus racemosus administration over placebo. Also, muscular endurance significantly increases with the administration of Asparagus racemosus in combination with resistance training, for instance as shown in the Example below, where bench press repetitions to failure significantly increased with Asparagus racemosus administration over placebo. Further, the combination of Asparagus racemosus administration with resistance training significantly increases the rate of change in increasing training loads as compared with placebo, for instance as shown in the below Example.

The below definitions and discussion are intended to guide understanding but are not intended to be limiting with regard to other disclosures in this application. References to percentage (%) in compositions of the present invention are to the % by weight of a given component to the total weight of the composition being discussed, also signified by “w/w”, unless stated otherwise.

“Administering”, “administration”, and the like, according to the present invention refer to providing a composition of the present invention to a subject so that the Asparagus racemosus may reach the subject's bloodstream and/or tissues and/or cells, preferably skeletal muscle tissues and cells and associated tissues and cells, and act on the tissues and cells, bloodstream and overall body of the subject to increase the subject's muscular strength and/or muscular endurance. Administration may be by the subject or by another. Administration may be oral, for instance in the form of a dietary supplement, and/or in a solid dosage form, preferably in a discrete dose unit, such as a capsule including for instance the 250 mg Asparagus racemosus capsule described below. Administration may also be through parenteral, intramuscular, transdermal, and other physiologically acceptable routes. In the below Example or as described elsewhere herein, supplementation with Asparagus racemosus is administration according to the present invention. A “subject” according to this invention is a human (male or female, adult or child) or other mammal such as a horse, dog, cat, cattle, zoo animals, or other mammals where increased muscle strength and endurance may be desired.

A “composition” of the present invention comprises Asparagus racemosus, preferably an extract of Asparagus racemosus such as an aqueous extract. A composition of the present invention may comprise, consist essentially of, or consist of, Asparagus racemosus or an extract of Asparagus racemosus. In the present invention, an “aqueous extract” is prepared by disrupting Asparagus racemosus from its natural state and treating the disrupted Asparagus racemosus with water or aqueous solution to form the aqueous extract. A “standardized aqueous extract” is an extract in which specific components have been identified and are present in a minimum or maximum amount or a specific range, so as to render the extract consistent at least with regard to those components from one batch to the next. In an embodiment, a composition according to the present invention comprises a standardized aqueous extract of Asparagus racemosus. In an embodiment, an Asparagus racemosus extract of this invention includes at least 40% saponins. A composition of the present invention may be a blend of Asparagus racemosus (e.g. standardized powdered aqueous extract of Asparagus racemosus) for instance with microcrystalline cellulose, croscarmellose sodium, and silicon dioxide as discussed below and as provided in the 250 mg capsules described in the below Example. A composition of the present invention may be formulated into nutraceutical or pharmaceutical dosage forms comprising for instance tablets, capsules, powders, liquids, chews, gummies, transdermals, injectables, dietary supplements, topical creams, lozenges, pills, and so forth. A composition of the present invention may further comprise one or more excipients, additives, and/or other substances, including for instance microcrystalline cellulose, croscarmellose sodium, magnesium stearate, and/or silicon dioxide. In the below Example or as described elsewhere herein, a supplement such as a 250 mg Asparagus racemosus capsule is a composition of the present invention.

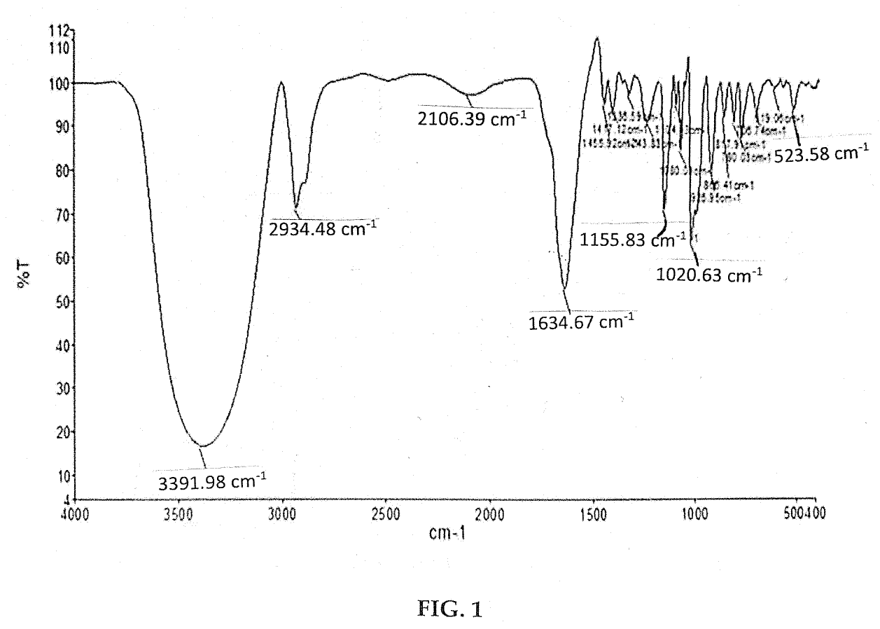

In an embodiment, a standardized, powdered, aqueous extract of Asparagus racemosus includes one or more of the following characteristics: Appearance: a free flowing powder, yellowish-brown in color, with a slightly bitter taste, and a water-soluble extractive value of about 85-95%, for instance 90-93%, for instance 91.38% (w/w). By assay, total saponins (by gravimetry) of the standardized powdered aqueous extract of Asparagus racemosus are at least 40% (w/w) of the extract, for instance about 40-60%, for instance 45-50%, for instance 48-49% (w/w), for instance 48.4% (w/w); total amino acids (including Arginine) of about 4-8%, for instance 5-6% (w/w), for instance 5.42%; moisture content (by Karl-Fischer titration) of about 1-8%, for instance 5-6%, for instance 5.80% (w/w); sulfated ash content of about 1-8%, for instance 3-4% (w/w), for instance 3.96%; particle size of the powdered extract passing through mesh #40 of 75-100%, for instance 100% and particle size passing through mesh #80 of about 80-100%, for instance 97-99%, for instance 98.30%; bulk density of about 0.2-0.5 grams/cubic cm, for instance 0.3-0.4 grams/cubic cm, for instance 0.384 gm/cc; and tapped density of about 0.3-0.6 grams/cubic cm, for instance 0.4-0.5 grams/cubic cm, for instance 0.425 gm/cc. In an embodiment, the standardized, powdered, aqueous extract of Asparagus racemosus is the Asparagus racemosus extract included in the 250 mg capsules used in the below Example. In an embodiment, Asparagus racemosus extract is stored in sealed containers at 15° C. to 25° C. In an embodiment, said storage avoids light. In an embodiment, the powdered Asparagus racemosus extract described above is stable for at least 3 years. An FTIR (Fourier Transform Infrared) chromatogram of a preferred, standardized, powdered, aqueous extract of Asparagus racemosus according to the present invention is shown in FIG. 1. FTIR readings at 19 points (reading left-to-right) are as follows: 1: 3391.98 cm⁻¹; 2: 2934.48 cm⁻¹; 3: 2106.39 cm⁻¹; 4: 1634.67 cm⁻¹; 5: 1455.92 cm⁻¹; 6: 1417.12 cm⁻¹; 7: 1336.59 cm⁻¹; 8: 1243.83 cm⁻¹; 9: 1155.83 cm⁻¹; 10: 1104.43 cm⁻¹; 11: 1080.09 cm⁻; 12: 1020.63 cm⁻¹; 13: 926.95 cm⁻; 14: 866.41 cm⁻¹; 15: 817.91 cm⁻¹; 16: 780.03 cm⁻¹; 17: 706.74 cm⁻¹; 18: 619.06 cm⁻¹; 19: 523.58 cm⁻¹. The standardized extract was a yellowish brown free flowing powder, with a slightly bitter taste, 91.38% (w/w) water-soluble extractive value, 48.40% (w/w) total saponins (by Gravimetry), 5.42% (w/w) total amino acids (including Arginine), 5.80% (w/w) moisture content (by Karl-Fischer titration), 3.96% (w/w) sulfated ash content, particle size such that 100% of particles pass through mesh #40 and 98.30% pass through mesh #80, bulk density of 0.384 gm/cc, and tapped density of 0.425 gm/cc.

In an embodiment, a capsule of the present invention includes Asparagus racemosus, for instance a standardized, powdered, aqueous extract of Asparagus racemosus such as identified above. In an embodiment, a capsule such as the 250 mg Asparagus racemosus capsule identified in the below Example is prepared as follows: standardized powdered aqueous extract of Asparagus racemosus is blended in amounts such as described in the below Example with microcrystalline cellulose, croscarmellose sodium, and silicon dioxide in a blender such as a V-blender, equipped with SIFT-N-BLEND, for instance for 5 minutes without using SIFT-N-BLEND and then 10 minutes using SIFT-N-BLEND at 1500 RPM. Next, magnesium stearate is added and blended for instance without SIFT-N-BLEND for 5 minutes. Then, the Asparagus racemosus blend is discharged from the blender into for instance a tared double poly bag lined HDPE drum, the bags tied shut, and the blend weighed and sealed into the drum. In an embodiment, the encapsulation of an Asparagus racemosus blend is in a room with humidity of not more than 40% RH (relative humidity). In an embodiment, a capsule contains about 316 mg of the Asparagus racemosus blend and weighs in total about 390 mg±3%. In an embodiment, the Asparagus racemosus blend is stored in original, sealed containers at 15° C. to 25° C. In an embodiment, said storage avoids light. In an embodiment, the powdered Asparagus racemosus blend described above is stable for at least 3 years. In an embodiment, a capsule containing 250 mg Asparagus racemosus in a blend as described above is white and opaque and in an embodiment comprises at least 30% (w/w) saponins.

A placebo capsule of the present invention may comprise one or more of microcrystalline cellulose, croscarmellose sodium, silicon dioxide, and magnesium stearate. For instance, a placebo capsule may include these ingredients in amounts defined in the Example below. A placebo capsule of the present invention may be prepared as follows: blend microcrystalline cellulose, croscarmellose sodium, and silicon dioxide in a blender such as a V-blender, equipped with SIFT-N-BLEND, for instance for 5 minutes without using SIFT-N-BLEND and then 10 minutes using SIFT-N-BLEND at 1500 RPM. Next, magnesium stearate is added and blended for instance without SIFT-N-BLEND for 5 minutes. Then, the blend is discharged from the blender into for instance a tared double poly bag lined HDPE drum, the bags tied shut, and the placebo blend weighed and sealed into the drum. In an embodiment, the encapsulation of a placebo blend is in a room with humidity of not more than 40% RH (relative humidity). In an embodiment, a capsule contains about 316 mg of the placebo blend and weighs in total about 390 mg±3%. In an embodiment, the placebo blend is stored in sealed containers at 15° C. to 25° C. In an embodiment, said storage avoids light.

In the present disclosure, an “effective amount” of Asparagus racemosus composition refers to an amount of Asparagus racemosus that, once administered to a subject, will reach the subject's bloodstream and/or bodily tissues and, in combination with resistance training, increase the subject's muscular strength and/or muscular endurance in skeletal muscle tissue and related tissues in the subject. In an embodiment, an effective amount of Asparagus racemosus is 500 mg/day, for instance in an adult human subject, when administered in combination with an effective amount of resistance training, for instance in keeping with the 8 week protocol discussed in the Example below. In an embodiment, an effective amount of Asparagus racemosus according to this invention is 50-1000 mg/day, for instance 50, 100, 200, 300, 400, 500, 600, 700, 800, 900, or 1000 mg/day, or any amount or range within said range. In an embodiment, an effective amount of Asparagus racemosus of this invention may be about 0.5 to about 20 mg/kg body weight, or another amount in view of the body weight of the subject. In an embodiment, an effective amount of Asparagus racemosus is administered to a subject daily. In an embodiment, the Asparagus racemosus may be administered for instance every other or every third day or fourth day so long as muscular endurance and strength are increased according to this invention. Effective amounts of Asparagus racemosus and resistance training together in combination increase muscular strength and/or muscular endurance according to methods of the present invention. An effective amount of resistance training may be for instance as shown in the Example below (3 times/week, about every other day), such as 1, 2, 3, or 4 sets of bench press repetitions per training session, and may include resistance training every day, or every other day, or every 3 days, or every 1-4 days, or every 1-7 days, so long as muscular strength and/or endurance increase with administration of Asparagus racemosus according to this invention. In an embodiment, administration of an effective amount of Asparagus racemosus to a subject and a subject's performance of an effective amount of resistance training may be combined together for instance for 1-6 days, 1-8 weeks, 4-16 weeks, or longer, to increase a subject's muscular strength and/or endurance. In an embodiment, said effective amount of resistance training is with a load corresponding to about 60-100% of the subject's 1RM.

A “dietary supplement” according to the present invention refers to a composition comprising Asparagus racemosus according to the present invention which is administered as an addition to a subject's diet, which is not a natural or conventional food, which when administered together with an effective amount of resistance training such as bench press training effectively increases muscular strength and/or muscular endurance in the subject for instance skeletal muscle tissue and related tissues of the subject's body over a period of time. In an embodiment, a dietary supplement containing an effective amount of Asparagus racemosus according to the present invention is administered orally. In an embodiment, the dietary supplement is administered daily; in an embodiment, the dietary supplement is administered daily for 1-2, 3-4, 5-6, 7-8, 1-8, 8 weeks, or more, or for another period of time according to the present invention. A dietary supplement may be formulated into various forms, such as powder, liquid, pill, capsule, or tablet, as discussed throughout this application.

“Resistance training” according to the present invention refers to causing a subject's skeletal muscles to contract against external resistance, such as by bench pressing weights, using dumbbells or a weight-lifting machine, or even an individual's own body weight. In an embodiment, resistance training is intended to increase muscular strength and/or muscular endurance. In the present invention, the “combination” of resistance training with Asparagus racemosus administration increases muscular strength and/or muscular endurance more than resistance training alone. One-repetition maximum strength and bench press repetitions to failure are in an embodiment part of a resistance training regimen designed to increase muscular strength and muscular endurance in a subject.

“Muscular strength” and “muscular endurance” refer to skeletal muscle and may include related tissues and are measured for instance by one-repetition maximum strength, bench press repetitions to failure, a rate of increase in training load, for instance as discussed in the below Example.

The present invention may be further understood in connection with the following Example and embodiments. The following non-limiting Example and embodiments described throughout this application are provided to illustrate the invention.

### Example

### A. Materials and Methods

### 1. Subjects

Twenty-eight college-aged men volunteered to participate in this study. Ten (Mean±SD; age=20.1±1.2 years; height=180.7±6.3 cm; body mass=88.2±12.8 kg) of the subjects were randomly assigned to the Asparagus racemosus group and eight (age=20.7±1.1 years; height=178.6±5.5 cm; body mass=81.4±11.0 kg) to the placebo group. A separate sample of ten additional men (age=22.0±2.3 years; height=177.8±6.9 cm; body mass=76.3±24.2 kg) was assigned to determine the test-retest reliability for the dependent variables and to calculate the minimal difference (MD) statistic [Weir, “Quantifying test-retest reliability using the intraclass correlation coefficient and the SEM. J. Strength Cond. Res. 19:231-240 (2005)]. All subjects were recreationally trained and had previously participated in resistance training exercises [Riebe et al., “ACSM's guidelines for exercise testing and prescription” American College of Sports Medicine (2018); ISBN 978-1-4963-3906-5]. The subjects had no known prior cardiovascular, metabolic, pulmonary, or musculoskeletal diseases. In addition, the subjects reported no use of any medication, nutritional product, dietary supplement or dietary program within the last month which would have interfered with the conduct of the study. The study was approved by the Institutional Review Board for Human Subjects at The University of Nebraska-Lincoln (IRB #20190219049FB). The subjects signed a written Informed Consent and health history questionnaire prior to participation.

### 2. Familiarization Visit

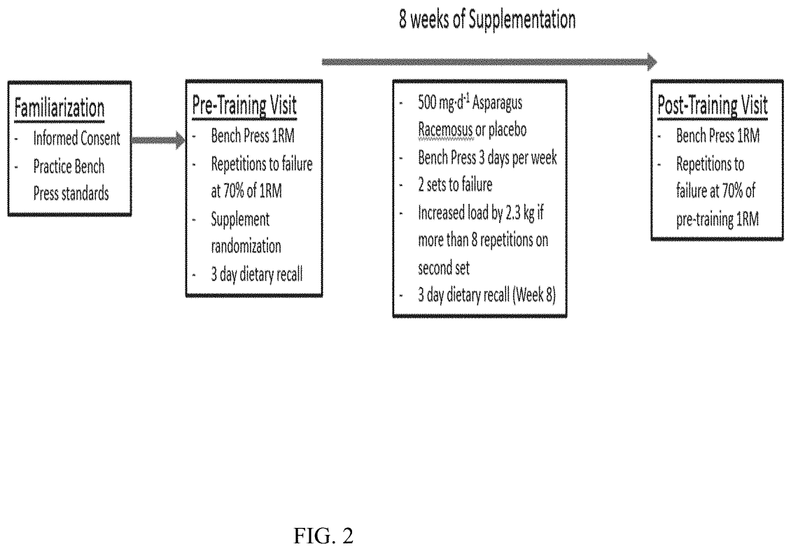

FIG. 2 shows a timeline for the Familiarization, Pre-Training, Training, and Post-Training Visits described below, along with the administration of 500 mg/day of Asparagus racemosus extract for 8 weeks.

The first laboratory visit consisted of an orientation session to familiarize the subjects with the testing and training protocols. During the orientation, the subjects performed submaximal bench press repetitions. The subjects then scheduled their pre-training test visit.

### 3. Pre-Training Test Visit

During the pre-training test visit, the subjects performed a one-repetition maximum strength test (1RM) and a bench press repetitions to failure test. The bench press was performed on a standard free-weight bench (Body Power, Williamsburg, Va., USA) with a traditional Olympic barbell. After an initial lift off from a spotter, the subjects were instructed to control the barbell down until it made contact with their chest, then lift the barbell back to a locked-out position in a controlled movement. During all bench press repetitions, a spotter was standing in position behind the bench to prepare to lift the barbell in the event the subject was unable to successfully complete a repetition. The 1RM was performed according to the guidelines established by the National Strength and Conditioning Association [Miller, “NSCA's Guide to Tests and Assessments” National Strength and Conditioning Association (US), Champaign, Ill. Human Kinetics (2012); ISBN 978-1-4925-8278-6]. Specifically, a light warm-up set was performed for 5-10 repetitions at 50% of their estimated 1RM, followed by 2-3 heavier warm up sets of 2-5 repetitions with loads increasing by 10-20% each set. The subjects then began completing trials of 1 repetition with increasing loads (5-10%) until they were no longer able to complete a single repetition. Verbal encouragement was provided, and two minutes of rest were allotted between trials. The highest load (kg) successfully lifted through the entire range of motion with proper technique was considered a 1RM. The 1RM was determined within 3 to 5 trials. After 10 minutes of rest, bench press repetitions to failure was assessed by subjects performing one set of as many repetitions to failure with a load corresponding to 70% of the 1RM established during the pre-training test visit. Failure was defined as the inability to complete a proper repetition [Miller, 2012]. The subjects were then randomized in either the 500 mg of Asparagus racemosus (Natreon Inc., New Brunswick, N.J., USA) (n=10) or 500 mg of placebo group (n=8) and instructed to consume 2 capsules (250 mg each) of their assigned supplement once a day for 8 weeks. The consumption of the capsules by the subjects is administration of the Asparagus racemosus according to the present invention. All capsules were very similar or identical in size and appearance. At the end of the pre-training test visit, subjects were instructed to complete and return a 3-day dietary recall form.

The 250 mg capsules contained a standardized, powdered, aqueous extract of Asparagus racemosus according to the present invention (Natreon Inc., New Brunswick, N.J.). The powdered extract is a free flowing powder, yellowish-brown in color, with a slightly bitter taste, standardized, with a water-soluble extractive value of 91.38% (w/w). Further, the extract was analyzed by assay, with total saponins (by gravimetry) present in an amount of 48.4% (w/w) of the extract, total amino acids (including Arginine) in an amount of 5.42% (w/w), moisture content (by Karl-Fischer titration) of 5.80% (w/w), sulfated ash content of 3.96% (w/w), particle size passing through mesh #40 (nominal sieve opening less than about 0.42 mm) of 100% w/w and particle size passing through mesh #80 (nominal sieve opening less than about 0.177 mm) of 98.30%, bulk density of 0.384 gm/cubic cm, and tapped density of 0.425 gm/cubic cm. In an embodiment, Asparagus racemosus is stored in original, sealed containers at 15° C. to 25° C. In an embodiment, said storage avoids light. In an embodiment, the powdered Asparagus racemosus extract described above is stable for up to 3 years.

In addition to 250 mg of the above standardized, powdered, aqueous extract of Asparagus racemosus (Natreon Inc., New Brunswick, N.J.), the 250 mg capsules contained the following: 50 mg microcrystalline cellulose, NF, 102 (VIVAPUR® 102, Mutchler Inc., Harrington Park, N.J.), 10 mg croscarmellose sodium, NF (VIVASOL® GF, JRS PHARMA LP, Patterson, N.Y.), 3 mg silicon dioxide, fumed, NF (AEROSIL®, Evonik Industries, Theodore, Ala.), 3 mg magnesium stearate, NF (Mutchler Inc., Harrington Park, N.J.). Empty capsules (Capsugel) were white and filled with 316 mg of a blend of the Asparagus racemosus extract and excipients as discussed above; total capsule weight including the capsule was 390 mg, with variation between capsules less than 2% by total weight of the capsule.

Placebo capsules included a placebo blend having the appearance of a white, odorless and tasteless free-flowing powder, said blend including the following excipients and no active ingredients: 300 mg microcrystalline cellulose, NF, 102 (VIVAPUR® 102, Mutchler Inc., Harrington Park, N.J.), 20 mg croscarmellose sodium, NF (VIVASOL® GF, JRS PHARMA LP, Patterson, N.Y.), 5 mg silicon dioxide, fumed, NF (AEROSIL®, Evonik Industries, Theodore, Ala.), 5 mg magnesium stearate, NF (Mutchler Inc., Harrington Park, N.J.). Empty capsules (Capsugel) were white; the filled capsule weight was 422 mg, with variation between capsules less than 2% by total weight of the capsule.

### 4. Training Visits

The training visits were supervised and performed 3 days per week for 8 consecutive weeks. Prior to the start of each training session, an investigator confirmed that the subjects were consuming their assigned supplement and asked whether they had experienced any adverse events. During each training visit, the subjects warmed up with 2 to 3 sets of low load resistance, then completed 2 sets of bench press to failure with loads initially corresponding to 80% of their 1RM. Verbal encouragement was provided during each set and two minutes of rest was allotted between sets. If a subject was able to perform more than 8 repetitions on the second set, 2.3 kg (about 5 pounds) was added to the start of the next training session. In the last week of the study, the subjects were instructed to complete and return a second 3-day dietary recall form.

### 5. Post-Training Test Visit

Following 8 weeks of training and supplementation, the subjects underwent a post-training test visit using the same testing protocol as the pre-training test visit. The post-training test visit included a 1RM bench press and bench press repetitions to failure at 70% of the pre-training 1RM.

### 6. Reliability of Bench Press 1RM and Endurance

Repeated measures of bench press 1RM and bench press repetitions to failure tests were assessed 2-7 days apart to determine test-retest reliability. The subjects (n=10) performed a 1RM, followed by bench press repetitions to failure and the protocols used were identical to those used during the pre-training and post-training test visits.

### 7. Statistical Analyses

Analyses of covariance (ANCOVA) were used to determine differences between the Asparagus racemosus and placebo groups for post-training bench press 1RM and repetitions to failure, covaried for pre-training values. Independent samples t-tests were used to compare the percent change in bench press 1RM and bench press repetitions to failure between the Asparagus racemosus and placebo groups. The training loads for each visit across the 8 weeks were log transformed and linear regression analyses were performed to compare the slope coefficients for the training load versus training visit relationship between the Asparagus racemosus and placebo groups. Separate 2 (Group [Asparagus racemosus and Placebo])×2 (Time [Pre-training and Post-training]) mixed factorial ANOVAs were used to compare total caloric and macronutrient intakes across the training period. Test-retest reliability for bench press 1RM and bench press repetitions to failure were assessed with a repeated measures ANOVA to identify systematic error and a 2,k model was used to determine the intraclass correlation coefficient (ICC) and minimal difference (MD) [Weir, 2005]. Effect sizes (η²ₚ and Cohen's d) were calculated for each comparison and an alpha of p<0.05 was considered statistically significant for all tests. The statistical analyses were performed using IBM SPSS v 25 (Armonk, N.Y., USA).

### B. Results

### 1. Reliability

The test-retest reliability for mean differences (systematic error), ICCs and MD for bench press 1RM and repetitions to failure were calculated using the 2,k model described by Weir [2005]. There were no mean differences between test versus retest of the bench press 1RM (104.8±22.7 vs 105.7±22.6; p=0.440, η²ₚ=0.067) and bench press repetitions to failure (13.4±1.4 vs 14.4±1.3; p=0.051, η²ₚ=0.360) (Table 1). 1RM and repetitions to failure were reliable measures of muscular strength and endurance, as shown for instance by the ICC values in Table 1.

TABLE 1
Test-retest reliability for bench press 1RM
and bench press repetitions to failure

| | Visit 1 | Visit 2 | | | | | | |
|---|---|---|---|---|---|---|---|---|
| | (Mean ± | (Mean ± | p- | | ICC | | CV | |
| | SD) | SD) | value | ICC | 95% CI | SEM | (%) | MD |
| 1RM (kg) | 104.8 ± | 105.6 ± | 0.440 | 0.994 | 0.98-0.99 | 2.53 | 2.4 | 7.01 |
| | 22.7 | 22.6 | | | | | | |
| Repetitions | 13.8 ± | 14.4 ± | 0.051 | 0.90 | 0.58-0.97 | 0.68 | 4.8 | 1.90 |
| to Failure | 1.5 | 1.3 | | | | | | |

CV (%) = coefficient of variation;
ICC = interclass correlation coefficient;
ICC 95% CI = interclass correlation coefficient 95% confidence interval;
MD = minimal difference needed to be considered real;
p-value = type I error rate for the one-way repeated measures analyses used to assess systematic variability;
SEM = standard error of the measurement.

### 2. Adverse Events, Adherence, Compliance, and Dietary Recall

The subjects reported no adverse or serious adverse events during the study and all of the subjects completed 24 bench press training sessions. The subjects reported consuming all daily doses of their assigned supplement throughout the 8 week training period. There were no significant interactions (p=0.149-0.812; η²ₚ=0.004-0.126) or main effects for Group (p=0.149-0.812; η²ₚ=0.004-0.126) or Time (p=0.225-0.970; η²ₚ=0.000-0.091) for total calories, carbohydrate, fat, and protein intake from their 3 day dietary recalls (Table 2).

TABLE 2
Total calories, carbohydrate, fat, and protein consumption across 3 days before and after
training (Mean ± SD)

| | Pre-training | | Post-training | |
|---|---|---|---|---|
| | Asparagus | | Asparagus | |
| Total | racemosus | Placebo | racemosus | Placebo |
| Calories (kcal) | 1623.1 ± 524.2 | 1639.0 ± 505.1 | 1544.1 ± 432.15 | 1953.2 ± 946.6 |
| Carbohydrate (g) | 154.1 ± 45.1 | 170.5 ± 55.5 | 138.6 ± 50.5 | 218.7 ± 151.2 |
| Fat (g) | 61.0 ± 23.5 | 54.4 ± 19.1 | 73.9 ± 45.4 | 65.5 ± 27.2 |
| Protein (g) | 112.8 ± 83.3 | 102.3 ± 35.0 | 97.8 ± 53.5 | 118.6 ± 45.6 |

### 3. Bench Press 1RM and Bench Press Repetitions to Failure

### Bench Press 1RM, Muscular Strength

Table 3 shows individual values as well as absolute and adjusted values for pre-training and post-training bench press 1RM. There was no significant (p=0.196, η²ₚ=0.109) difference for the adjusted mean bench press 1RM between the Asparagus racemosus (106.1±5.1 kg) and placebo (102.7±5.1 kg) groups when covaried for pre-training values (Table 3). The results of the independent samples t-test demonstrated that the Asparagus racemosus group had a significantly (p=0.048) greater percent increase in bench press 1RM (14.3±7.7%) compared to the placebo group (7.8±4.5%; d=1.06) (Table 3). The minimal difference (MD) for a change to be real for bench press 1RM of an individual subject was 7.01 kg, based on the reliability data (Table 1). In the Asparagus racemosus group, 8 of the 10 subjects (80%) exceeded the MD and increased their 1RM bench press and thereby their muscular strength, while 4 of the 8 subjects (50%) in the placebo group exceeded the MD and increased their 1RM bench press and thereby their muscular strength. Raw data representing the 24 training sessions (3 per week over 8 weeks) is represented in Table 3A.

TABLE 3
Pre-Training and Post-Training Bench Press 1RM
(Individual, Absolute, and Adjusted Values (Mean ± SD))

| | | | | |
|---|---|---|---|---|
| | | | Absolute | Percent |
| | Pre-Training | Post-Training | Change | Change |
| Subject | 1RM (kg) | 1RM (kg) | (kg) | (%) |

Asparagus racemosus Group

| | | | | |
|---|---|---|---|---|
| 1 | 70.3 | 83.9 | 13.6* | 19.3 |
| 2 | 79.4 | 93.0 | 13.6* | 17.1 |
| 3 | 124.7 | 124.7 | 0.0 | 0.0 |
| 4 | 83.9 | 102.1 | 18.2* | 21.6 |
| 5 | 115.7 | 124.7 | 9.1* | 7.8 |
| 6 | 102.1 | 115.7 | 13.6* | 13.3 |
| 7 | 52.2 | 65.8 | 13.6* | 26.1 |
| 8 | 90.7 | 97.5 | 6.8 | 7.5 |
| 9 | 102.1 | 115.7 | 13.6* | 13.3 |
| 10 | 79.4 | 93.0 | 13.6* | 17.1 |
| Mean | 90.0 ± 21.7 | 101.6 ± 18.9 | 11.6 ± 5.1 | 14.3 ± 7.7** |

| | |
|---|---|
| Adjusted | 106.1 ± 5.1 |
| Mean | |

Placebo Group

| | | | | |
|---|---|---|---|---|
| 1 | 83.9 | 93.0 | 9.1* | 10.8 |
| 2 | 79.4 | 81.7 | 2.3 | 2.9 |
| 3 | 65.8 | 68.0 | 2.3 | 3.5 |
| 4 | 142.9 | 154.2 | 11.3* | 7.9 |
| 5 | 102.1 | 115.7 | 13.6* | 13.3 |
| 6 | 111.1 | 117.9 | 6.8 | 6.1 |
| 7 | 97.5 | 111.1 | 13.6* | 14.0 |
| 8 | 120.2 | 124.7 | 4.5 | 3.8 |
| Mean | 100.4 ± 24.6 | 108.3 ± 26.9 | 7.9 ± 4.7 | 7.8 ± 4.5 |

| | |
|---|---|
| Adjusted | 102.7 ± 5.1 |
| Mean | |

*Minimal difference (MD) value for a change to be “real” for an individual subject in bench press 1RM was 7.01 kg, based on reliability data in Table 1.
**Percent change (%) in 1RM bench press for the Asparagus racemosus group was greater than the placebo group at p = 0.048.
Adjusted Mean ± SD post-test values were covaried for pre-training values for the Asparagus racemosus group and the placebo group.

TABLE 3A
1RM DAILY TRAINING LOADS (raw data, weight in pounds)

| | | (weight in pounds) | | | | | | | |
|---|---|---|---|---|---|---|---|---|---|
| | | Week 1 | | | Week 2 | | | Week 3 | |
| | | Training | Training | Training | Training | Training | Training | Training | Training |
| Subject | Group | Visit_1 | Visit_2 | Visit_3 | Visit_4 | Visit_5 | Visit_6 | Visit_7 | Visit_8 |
| 1 | A. | 115 | 115 | 115 | 115 | 115 | 115 | 120 | 125 |
| | racemosus | | | | | | | | |
| 2 | A. | 140 | 140 | 140 | 140 | 140 | 140 | 145 | 145 |
| | racemosus | | | | | | | | |
| 3 | A. | 220 | 220 | 220 | 225 | 230 | 230 | 230 | 230 |
| | racemosus | | | | | | | | |
| 4 | A. | 150 | 150 | 150 | 150 | 150 | 150 | 150 | 150 |
| | racemosus | | | | | | | | |
| 5 | A. | 180 | 185 | 205 | 210 | 210 | 210 | 215 | 225 |
| | racemosus | | | | | | | | |
| 6 | A. | 180 | 180 | 180 | 180 | 185 | 190 | 195 | 195 |
| | racemosus | | | | | | | | |
| 7 | A. | 90 | 90 | 90 | 90 | 90 | 90 | 90 | 90 |
| | racemosus | | | | | | | | |
| 8 | A. | 160 | 165 | 170 | 170 | 170 | 170 | 170 | 170 |
| | racemosus | | | | | | | | |
| 9 | A. | 180 | 180 | 180 | 180 | 180 | 180 | 185 | 185 |
| | racemosus | | | | | | | | |
| 10 | A. | 140 | 140 | 140 | 140 | 145 | 145 | 150 | 155 |
| | racemosus | | | | | | | | |
| | AVERAGE | 155.5 | 156.5 | 159 | 166 | 161.5 | 162 | 165 | 167 |
| 1 | Placebo | 150 | 150 | 150 | 150 | 150 | 150 | 150 | 150 |
| 2 | Placebo | 140 | 145 | 150 | 150 | 150 | 150 | 150 | 150 |
| 3 | Placebo | 115 | 115 | 115 | 115 | 115 | 115 | 115 | 115 |
| 4 | Placebo | 250 | 250 | 250 | 250 | 255 | 255 | 255 | 260 |
| 5 | Placebo | 180 | 180 | 180 | 180 | 185 | 190 | 190 | 190 |
| 6 | Placebo | 195 | 200 | 200 | 200 | 205 | 210 | 210 | 210 |
| 7 | Placebo | 170 | 170 | 170 | 170 | 170 | 170 | 170 | 170 |
| 8 | Placebo | 215 | 215 | 215 | 215 | 220 | 220 | 220 | 220 |
| | AVERAGE | 176.875 | 178.125 | 178.75 | 178.75 | 181.25 | 182.5 | 182.5 | 183.125 |
| | | | Week 4 | | | Week 5 | | | Week 6 |
| | | Training | Training | Training | Training | Training | Training | Training | Training |
| Subject | Group | Visit_9 | Visit_10 | Visit_11 | Visit_12 | Visit_13 | Visit_14 | Visit_15 | Visit_16 |
| 1 | A. | 130 | 130 | 135 | 140 | 145 | 145 | 150 | 150 |
| | racemosus | | | | | | | | |
| 2 | A. | 150 | 150 | 155 | 160 | 165 | 165 | 170 | 170 |
| | racemosus | | | | | | | | |
| 3 | A. | 230 | 230 | 235 | 235 | 235 | 235 | 235 | 235 |
| | racemosus | | | | | | | | |
| 4 | A. | 150 | 150 | 155 | 155 | 155 | 155 | 160 | 160 |
| | racemosus | | | | | | | | |
| 5 | A. | 225 | 225 | 230 | 230 | 235 | 235 | 235 | 235 |
| | racemosus | | | | | | | | |
| 6 | A. | 195 | 195 | 200 | 205 | 205 | 205 | 205 | 205 |
| | racemosus | | | | | | | | |
| 7 | A. | 90 | 95 | 95 | 105 | 105 | 105 | 105 | 105 |
| | racemosus | | | | | | | | |
| 8 | A. | 170 | 175 | 175 | 175 | 175 | 175 | 175 | 175 |
| | racemosus | | | | | | | | |
| 9 | A. | 185 | 190 | 190 | 195 | 195 | 195 | 195 | 195 |
| | racemosus | | | | | | | | |
| 10 | A. | 155 | 155 | 165 | 165 | 165 | 165 | 165 | 165 |
| | racemosus | | | | | | | | |
| | AVERAGE | 168 | 169.5 | 173.5 | 176.5 | 178 | 178 | 179.5 | 179.5 |
| 1 | Placebo | 150 | 150 | 150 | 155 | 155 | 155 | 155 | 155 |
| 2 | Placebo | 150 | 150 | 150 | 150 | 150 | 150 | 150 | 150 |
| 3 | Placebo | 115 | 115 | 115 | 120 | 120 | 125 | 125 | 125 |
| 4 | Placebo | 260 | 265 | 265 | 265 | 270 | 270 | 275 | 275 |
| 5 | Placebo | 190 | 190 | 190 | 190 | 195 | 195 | 195 | 195 |
| 6 | Placebo | 210 | 215 | 215 | 215 | 215 | 220 | 220 | 225 |
| 7 | Placebo | 170 | 170 | 170 | 170 | 170 | 170 | 170 | 175 |
| 8 | Placebo | 220 | 220 | 220 | 225 | 225 | 225 | 225 | 225 |
| | AVERAGE | 183.125 | 184.375 | 184.375 | 186.25 | 187.5 | 188.75 | 189.375 | 190.625 |
| | | | | Week 7 | | | Week 8 | | |
| | | Training | Training | Training | Training | Training | Training | Training | Training |
| Subject | Group | Visit_17 | Visit_18 | Visit_19 | Visit_20 | Visit_21 | Visit_22 | Visit_23 | Visit_24 |
| 1 | A. | 155 | 155 | 155 | 155 | 155 | 155 | 155 | 155 |
| | racemosus | | | | | | | | |
| 2 | A. | 170 | 175 | 175 | 175 | 173 | 175 | 175 | 175 |
| | racemosus | | | | | | | | |
| 3 | A. | 235 | 235 | 235 | 235 | 235 | 235 | 235 | 235 |
| | racemosus | | | | | | | | |
| 4 | A. | 165 | 170 | 175 | 180 | 185 | 185 | 190 | 190 |
| | racemosus | | | | | | | | |
| 5 | A. | 235 | 235 | 235 | 235 | 235 | 240 | 240 | 240 |
| | racemosus | | | | | | | | |
| 6 | A. | 210 | 210 | 210 | 210 | 210 | 210 | 210 | 210 |
| | racemosus | | | | | | | | |
| 7 | A. | 110 | 110 | 115 | 115 | 115 | 115 | 115 | 115 |
| | racemosus | | | | | | | | |
| 8 | A. | 180 | 180 | 180 | 180 | 180 | 180 | 180 | 180 |
| | racemosus | | | | | | | | |
| 9 | A. | 200 | 200 | 200 | 200 | 200 | 205 | 205 | 205 |
| | racemosus | | | | | | | | |
| 10 | A. | 165 | 165 | 165 | 165 | 165 | 170 | 170 | 170 |
| | racemosus | | | | | | | | |
| | AVERAGE | 182.5 | 182.5 | 184.5 | 185 | 185.5 | 187 | 187.5 | 187.5 |
| 1 | Placebo | 160 | 165 | 165 | 165 | 165 | 165 | 165 | 165 |
| 2 | Placebo | 150 | 150 | 150 | 150 | 150 | 150 | 155 | 155 |
| 3 | Placebo | 125 | 125 | 125 | 125 | 125 | 130 | 130 | 130 |
| 4 | Placebo | 275 | 275 | 275 | 275 | 275 | 275 | 275 | 275 |
| 5 | Placebo | 195 | 195 | 195 | 195 | 195 | 195 | 195 | 195 |
| 6 | Placebo | 225 | 225 | 225 | 225 | 230 | 230 | 230 | 230 |
| 7 | Placebo | 180 | 180 | 185 | 185 | 185 | 190 | 195 | 195 |
| 8 | Placebo | 225 | 225 | 225 | 225 | 230 | 230 | 230 | 230 |
| | AVERAGE | 191.875 | 192.5 | 193.125 | 193.125 | 194.375 | 195.625 | 196.88 | 196.88 |

### Bench Press Repetitions to Failure, Muscular Endurance

The Asparagus racemosus group demonstrated significantly (p=0.044) greater adjusted bench press repetitions to failure (17.5±2.2 repetitions) than the placebo group (15.2±2.2; η²ₚ=0.243) when covaried for pre-training values (Table 4). There was no significant difference (p=0.058) in the percent change for bench press repetitions to failure between the Asparagus racemosus (33.2±27.8%) and placebo groups (12.2±11.1%; d=1.00) (Table 4). The MD for the change to be real for bench press repetitions to failure was 1.9 repetitions, based on the reliability data (Table 1). In the Asparagus racemosus group, 8 of the 10 subjects (80%) exceeded the MD and increased their repetitions to failure and thereby their muscular endurance, while 3 of the 8 subjects (30%) in the placebo group exceeded the MD and increased their repetitions to failure and thereby their muscular endurance.

TABLE 4
Pre-Training And Post-Training Bench Press Repetitions To Failure
(Individual, absolute, and adjusted Mean ± SD values)

| | | | | |
|---|---|---|---|---|
| | | | Absolute | |
| | Pre-Training | Post-Training | Change | Percent |
| Subject | Repetitions | Repetitions | (repetitions) | Change (%) |

Asparagus racemosus Group

| | | | | |
|---|---|---|---|---|
| 1 | 12 | 20 | 8 | 66.7 |
| 2 | 14 | 15 | 1 | 7.1 |
| 3 | 14 | 13 | 1 | 7.0 |
| 4 | 13 | 18 | 5* | 38.5 |
| 5 | 15 | 18 | 3* | 20.0 |
| 6 | 15 | 17 | 2* | 13.3 |
| 7 | 10 | 19 | 9* | 90.0 |
| 8 | 15 | 19 | 4* | 26.7 |
| 9 | 11 | 16 | 5 | 45.5 |
| 10 | 17 | 20 | 3* | 17.6 |
| Mean | 13.6 ± 2.1 | 17.5 ± 2.3 | 4.1 ± 2.7 | 33.2 ± 27.4 |

| | |
|---|---|
| Adjusted | 17.5 ± 2.2** |
| Mean | |

Placebo Group

| | | | | |
|---|---|---|---|---|
| 1 | 14 | 14 | 0 | 0.0 |
| 2 | 14 | 14 | 0 | 0.0 |
| 3 | 11 | 12 | 1 | 9.1 |
| 4 | 11 | 14 | 3* | 27.3 |
| 5 | 14 | 15 | 1 | 7.1 |
| 6 | 14 | 15 | 1 | 7.1 |
| 7 | 15 | 19 | 4* | 26.7 |
| 8 | 15 | 18 | 3* | 20.0 |
| Mean | 13.5 ± 1.6 | 15.1 ± 2.3 | 1.6 ± 1.5 | 12.2 ± 11.1 |

| | |
|---|---|
| Adjusted | 15.2 ± 2.2 |
| Mean | |

*Minimal difference (MD) value for a change to be “real” for an individual subject in bench press repetitions to failure was 1.90 repetitions, based on reliability data in Table 1.
**Adjusted post-training repetitions to failure for the Asparagus racemosus group was greater than the placebo group at p = 0.044.
Adjusted Mean ± SD post-test values were covaried for pre-training values for the Asparagus racemosus group and the placebo group.

### 4. Training Load

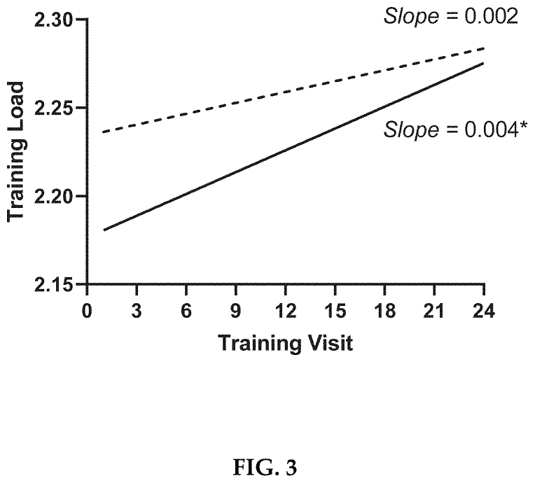

Over 8 weeks of training, the rate of change in the bench press training loads was significantly (p<0.001) greater for the Asparagus racemosus group (slope=0.004±0.0005) than the placebo group (slope=0.002±0.0002) (FIG. 3). FIG. 3 shows regression analyses of the log-transformed training loads for the Asparagus racemosus group (-) and the placebo group (- - -). The “*” in FIG. 3 indicates the slope coefficient for the Asparagus racemosus group was significantly (p<0.001) greater than the placebo group.

### C. Discussion

Overall, the administration of Asparagus racemosus in a resistance training program enhanced percent increases in muscular strength and improved muscular endurance performance, increasing muscular strength and endurance in keeping with this invention.

Administering 500 mg·d⁻¹ of Asparagus racemosus, in combination with bench press training 3 days per week, resulted in a 6.5% greater increase in muscular strength on average as measured and shown by increased bench press 1RM (14.3±7.7%) over placebo (7.8±4.5%), and individual increases of about 7-30% as compared with placebo (about 3-14%; see Table 3); a greater mean increase in muscular endurance as measured and shown by increased bench press repetitions to failure (17.5±2.2 repetitions) compared with placebo (15.2±2.2 repetitions), and individual increases of about 7-90% compared with about 0-27% increase in the placebo subjects (see Table 4); and greater increases in muscular strength (80% vs. 50% placebo) and endurance (80% vs. 38% placebo). Furthermore, daily supplementation with Asparagus racemosus resulted in a greater rate of increase in training load throughout the 8 weeks compared to the placebo group, for instance as shown in Table 3A, an average increase in training loads of about 32 pounds (Asparagus racemosus group; 187.5-155.5) as compared with about 20 pounds (placebo group; 196.9-176.9). Administration of Asparagus racemosus facilitated greater increases in training loads throughout the 8 weeks of resistance training, contributing to the improvements in muscular strength and endurance.

The results of the test-retest reliability data demonstrated that the bench press 1RM and bench press repetitions to failure were highly reliable measures of muscular strength and endurance (Table 1). Calculation of the MD values from the reliability analyses in the present study indicated that for individual subjects, training-induced changes in bench press 1RM and bench press repetitions to failure of 7.0 kg and 1.9 repetitions, respectively, were required to be considered “real” [Weir, 2005]. A recent review [Roberts et al., “Physiological Differences Between Low Versus High Skeletal Muscle Hypertrophic Responders to Resistance Exercise Training: Current Perspectives and Future Research Directions” Front. Physiol. 2018, 9] has characterized individuals as high or low responders to training-induced adaptations to resistance training. Thus, it is important to examine the training-induced responses of individual subjects, as well as group mean responses. The findings of the present study demonstrated no mean difference between groups for improvements in absolute bench press 1RM (Table 3), but a greater mean improvement in absolute bench press repetitions to failure (Table 4) for the Asparagus racemosus group than the placebo group. On an individual basis, 80% of the subjects in the Asparagus racemosus group exceeded the MD of 7.01 kg needed to be considered a real change for the bench press 1RM, while only 50% of subjects exceeded the MD for the placebo group. For bench press repetitions to failure, 80% of the subjects in the Asparagus racemosus group exceeded the MD of 1.9 repetitions to failure needed to be considered a real change, compared to 38% of the subjects in the placebo group. Calculation of the MD in the present study allowed for a practical interpretation of the training-induced changes in muscular strength and endurance on a subject by subject basis [Weir, 2005]. Thus, the results of the present study demonstrated that supplementation with Asparagus racemosus elicited greater improvements for bench press 1RM and bench press repetitions to failure compared to placebo on an individual subject basis that were only partially reflected in the group mean analyses (Tables 3 and 4).

In the present study, supplementation with Asparagus racemosus over 8 weeks improved bench press training loads by eliciting a greater rate of increase in the training loads compared to the placebo group (FIG. 3). The greater rate of increase in the training load for the Asparagus racemosus group likely contributed the greater mean increase in bench press repetitions to failure, as well as the greater percentage of “real” individual increases in both bench press 1RM and bench press repetitions to failure compared to the placebo group. In an embodiment, the greater rate of increase in training loads over a longer period of resistance training plus supplementation with Asparagus racemosus will lead to greater mean and individual increases in muscular strength and endurance.

No previous studies have examined the influence of supplementation with Asparagus racemosus on exercise performance. Without being bound by theory, in the Ayurvedic tradition, Asparagus racemosus has been utilized as an adaptogen, in part, due to its antioxidant properties [Rege, 1999; Alok, 2013; Bopana, 2007; Kamat et al., “Antioxidant properties of Asparagus racemosus against damage induced by gamma-radiation in rat liver mitochondria” J. Ethnopharmacol. 71:425-435 (2000)]. Reactive oxygen species, such as those produced during exercise [Jackson, “Control of reactive oxygen species production in contracting skeletal muscle” Antioxid. Redox Signal. 15:2477-2486 (2011)], undergo oxidative reactions with cellular mechanisms that can impair muscle function and growth through the disruption of cellular functions such as myofibrillar calcium dynamics and gene transcription [Moylan et al., “Oxidative stress, chronic disease, and muscle wasting” Muscle Nerve 35:411-429 (2007); Reid, “Reactive Oxygen Species as Agents of Fatigue” Med. Sci. Sports Exerc. 48:2239-2246 (2016)]. Enzymatic and nonenzymatic antioxidants function to buffer, scavenge, and minimize the deleterious effects of reactive oxygen species [Patlevič et al., “Reactive oxygen species and antioxidant defense in human gastrointestinal diseases” Integr. Med. Res. 5:250-258 (2016); Powers et al., “Exercise-Induced Oxidative Stress: Cellular Mechanisms and Impact on Muscle Force Production” Physiol. Rev. 88:1243 (2008)]. Wiboonpun et al. [“Identification of antioxidant compound from Asparagus racemosus” Phytother. Res. 18:771-773 (2004)] reported the presence of antioxidants in Asparagus racemosus including asparagamine A, racemosol, and racemofuran. Furthermore, Kamat et al. (2000) demonstrated that supplementation with Asparagus racemosus attenuated mitochondrial oxidative stress elicited by radiation-induced reactive oxygen species in the rat model. Although there is conflicting evidence [Ismaeel et al., “Resistance Training, Antioxidant Status, and Antioxidant Supplementation” Int. J. Sport Nutr. Exerc. Metab. 29:539-547 (2019); Merry et al., “Do antioxidant supplements interfere with skeletal muscle adaptation to exercise training?” J. Physiol. 594:5135-5147 (2016)], the use of antioxidants as an ergogenic aid has demonstrated improvements in exercise performance [McKenna et al., “N-acetylcysteine attenuates the decline in muscle Na+,K+-pump activity and delays fatigue during prolonged exercise in humans” J. Physiol. 576:279-288 (2006); Levers et al., “Effects of powdered Montmorency tart cherry supplementation on an acute bout of intense lower body strength exercise in resistance trained males” J. Int. Soc. Sports Nutr. 12:41 (2015); Aguiló et al., “Antioxidant diet supplementation enhances aerobic performance in amateur sportsmen” J. Sports Sci. 25:1203-1210 (2007); Bowtell et al., “Montmorency cherry juice reduces muscle damage caused by intensive strength exercise” Med. Sci. Sports Exerc. 43:1544-1551 (2011)]. For example, Aguilo et al. [2007] reported that the antioxidant effects of 90 days of vitamin E and β-carotene supplementation during duathlon training resulted in improved lactate buffering and utilization. Bowtell et al. [2011] found that the antioxidant effects associated with 7 days of Montmorency cherry juice concentrate supplementation led to improved force recovery and lower creatine kinase activity 24 and 48 hours following a muscle damaging protocol that included 10 sets of 10 repetitions of leg extensions at 80% of 1RM. Levers et al. [2015] showed that the antioxidant effects of 10 days of powdered tart cherry supplementation reduced the perception of muscle soreness and serum creatinine concentrations following 10 sets of 10 repetitions of back squat at 70% of 1RM. During submaximal cycle ergometry, McKenna [2006] reported a greater time to exhaustion and improved plasma potassium regulation with continuous infusion of the antioxidant N-acetylcysteine. In the present Example, again without being bound by theory, exercise-induced oxidative stress [Reid, 2016] may have been mitigated by the antioxidant properties of Asparagus racemosus, which enhanced muscle recovery and reduced muscle soreness on a day to day basis that contributed to the greater rate of increase in training load throughout the 8 weeks of training. The antioxidant effects of Asparagus racemosus may have also contributed to the mean improvements in muscular endurance and the higher percentage of individual subjects that exhibited real training-induced increases in bench press 1RM and bench press repetitions to failure.

Limitations of the present Example include the low mean total caloric and macronutrient consumption values from the 3-day dietary recall when compared to the dietary recommendations for the subjects' age demographic [Institute of Medicine (U.S.), “Dietary reference intakes for energy, carbohydrate, fiber, fat, fatty acids, cholesterol, protein, and amino acids” National Academies Press: Washington, D.C. (2005); ISBN 978-0-309-08525-0]. Previous studies, however, have demonstrated dietary recalls are subject to systematic underreporting of nutritional intake [Institute of Medicine (US) Committee on Dietary Risk Assessment in the WIC Program Food-Based Assessment of Dietary Intake. National Academies Press (US), Washington (DC) (2002) 5; DOI: 10.17226/10342]. Furthermore, the present study utilized only the bench press to assess muscular strength and endurance. In addition, the subjects self reported their adherence to daily supplementation, but were not supervised in the taking of their assigned supplement.

The present methods increase muscular strength and muscular endurance with the combined administration of Asparagus racemosus and performance of resistance training. During 8 weeks of resistance training, supplementation with 500 mg·d⁻¹ of Asparagus racemosus elicited greater mean percentage and individual increases in bench press 1RM, mean and individual increases in bench press repetitions to failure, and a greater rate of increase in bench press training loads compared to placebo.

The use of the terms “a,” “an,” “the,” and similar referents in the context of describing the present invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments, the values may range in value above or below the stated value in a range of approximately ±5%; in other embodiments, the values may range in value above or below the stated value in a range of approximately ±2%; in other embodiments, the values may range in value above or below the stated value in a range of approximately ±1%. The preceding ranges are intended to be made clear by context, and no further limitation is implied. All method steps described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise stated. No language in the specification should be construed as indicating any non-claimed element as essential to the practice of the invention.

While in the foregoing specification the present invention has been described in relation to certain embodiments thereof, and many details have been put forth for the purposes of illustration, it will be apparent to those skilled in the art that the invention is susceptible to additional embodiments and that certain of the details described herein can be varied considerably without departing from the basic principles of the invention.

The present invention may be embodied in other specific forms without departing from the spirit or essential attributes thereof, and, accordingly, reference should be made to the appended claims, rather than to the foregoing specification, as indicating the scope of the invention.

1. A method of increasing muscular strength and/or muscular endurance in a subject comprising the steps of:
(a) providing a composition comprising an Asparagus racemosus extract;
(b) administering an effective amount of the composition to the subject to deliver the Asparagus racemosus to the subject's bloodstream and bodily tissues;
wherein the subject performs an effective amount of resistance training; and
wherein the Asparagus racemosus acts in said bloodstream and/or bodily tissues in combination with said resistance training to increase the subject's muscular strength and muscular endurance.

2. The method of claim 1, wherein said increase in muscular strength and/or muscular endurance is increased one-repetition maximum (1RM) strength.

3. The method of claim 2, wherein said increased 1RM strength is an increase of about 5% to about 50% after administration of an effective amount of Asparagus racemosus and resistance training.

4. The method of claim 1, wherein said Asparagus racemosus is administered daily to the subject.

5. The method of claim 4, wherein said resistance training is performed at least 3 times per week.

6. The method of claim 1, wherein said increase in muscular strength and/or muscular endurance is increased bench press repetitions to failure.

7. The method of claim 6, wherein said increased bench press repetitions to failure is an increase on average of 2 or more repetitions before failure.

8. The method of claim 6, wherein said Asparagus racemosus is administered daily to the subject.

9. The method of claim 8, wherein said resistance training is performed at least 3 times per week.

10. The method of claim 1, wherein said increase in muscular strength and/or muscular endurance is increased rate of change in bench press training loads.

11. The method of claim 6, wherein said increased rate of change in bench press training loads is an increase of at least 10 pounds within 3 weeks of beginning administration of Asparagus racemosus in combination with resistance training.

12. The method of claim 10, wherein said Asparagus racemosus is administered daily to the subject.

13. The method of claim 12, wherein said resistance training is performed at least 3 times per week.

14. The method of claim 1, wherein said composition is a dietary supplement.
